(19) United States
(12) Patent Application Publication
(10) Pub. No.: US 2022/0319703 A1
(43) Pub. Date: Oct. 6, 2022

(54) SYSTEM AND METHOD TO PREDICT REPRODUCIBILITY OF CLINICAL BENEFITS OF A NEW DRUG

(71) Applicant: Actu-Real, Inc., Alpharetta, GA (US)

(72) Inventors: Pierre Emile ETIENNE, Enosburg Falls, VT (US); Chitra Lele, Pune (IN)

(21) Appl. No.: 17/581,339

(22) Filed: Jan. 21, 2022

Related U.S. Application Data
(60) Provisional application No. 63/171,366, filed on Apr. 6, 2021.

(51) Int. Cl. G16H 50/20; G16H 10/60; G16H 10/20; G16H 20/10

(52) U.S. Cl. CPC G16H 50/20; G16H 10/60; G16H 10/20; G16H 20/10

(57) ABSTRACT

A computer-implemented method of predicting reproducibility of clinical benefits of a new drug after its launch is provided. The method includes accessing standard of care (SOC) medical records and identifying in SOC records a plurality of patient variables that are statistically related to one or more endpoints reflected in clinical benefits of the new drug as presented in the package insert information of the drug. The method also includes estimating the strength of identified relationships between the patient variables and the one or more endpoints reflected in the clinical benefits of the new drug using an analytical model. The method includes oversampling the SOC medical records to create a first set of proxy medical records and combining the first set of proxy medical records with the SOC records to create proxy medical records. The method further includes creating a subset of proxy medical records that meet eligibility criteria of registration trials participants and reproducing the clinical benefits of the new drug in the subset of proxy medical records that meet the eligibility criteria of registration trials participants by adjusting one or more variables related to the clinical benefits in the analytical model and recorded in phase 3 registration trials. The method further includes applying the adjustments to the proxy medical records to generate post launch clinical benefits that persist after dilution of those adjustments by variables that were not represented in the phase 3 registration trials and accessing medical payment records for translating the post launch clinical benefits that persist into health outcome and health resource utilization.

### PRIORITY STATEMENT

The present application hereby claims priority to U.S. Provisional Application No. 63/171,366 filed on 6 Apr. 2021, the entire contents of which are hereby incorporated herein by reference.

### FIELD

The present invention relates to a system and method to predict reproducibility of clinical benefits of a new drug and, more particularly, to techniques relating to determination of value of a new drug at launch.

### BACKGROUND

Typically, the value of a new drug or biologic is a function of several variables such as improved health outcomes (e.g., increased survival rate, improved health, reduced health resource utilization and reduction or postponement of adverse experiences) and is typically evaluated in comparison to standard of care treatment.

Certain techniques are employed to estimate the value of new drugs. Although the benefits of improved health upon usage of the drugs may be difficult to quantify, reduction in health care resource utilization that may be adjusted for survival may be measurable. Such parameters may also serve as proxy for improved health of patients using the drug.

One of the issues faced by pharma sponsors and payers at the time of launch of a new drug is determination of the value of the drug. In practice, it is difficult to estimate the value of the drug by extrapolating clinical results from randomized clinical trials (RCTs), used for registration studies, to the post-launch real world. As a result, it is not possible to anticipate resulting reductions in health care resources utilization by the respective drug utilization.

Sponsors have to use inadequate or costly techniques to reduce the uncertainty around the drug value because currently there are no better options available. In some cases, they may choose to perform post-launch comparisons between clinical results obtained with the new drug and clinical results obtained concurrently or historically with a standard of care drug (SOC). In other cases, sponsors may choose to conduct comparative studies that resemble randomized clinical trials (RCTs) in some respects but on a population no longer restricted by inclusion and exclusion criteria applied in phase 3 (P3) trials. These trials are expensive and may be time consuming. However, postponing or cancelling them has a cost too, delaying access to data that would reduce the uncertainty around the value of the drug.

These problems make value-based-pricing (VBP) and risk sharing agreements (RSAs) (including formulary entry negotiations) between sponsors and payers exceedingly difficult.

### SUMMARY

The following summary is illustrative only and is not intended to be in any way limiting. In addition to the illustrative aspects, example embodiments, and features described, further aspects, example embodiments, and features will become apparent by reference to the drawings and the following detailed description.

Briefly, according to an example embodiment, a computer-implemented method of predicting reproducibility of clinical benefits of a new drug after its launch is provided. The method includes accessing standard of care (SOC) medical records and identifying in SOC records a plurality of patient variables that are statistically related to one or more endpoints reflected in clinical benefits of the new drug as presented in the package insert information of the drug or published results from RCTs. The method also includes estimating the strength of identified relationships between the patient variables and the one or more endpoints reflected in the clinical benefits of the new drug using an analytical model. The method includes oversampling the SOC medical records to create a first set of proxy medical records and combining the first set of proxy medical records with the SOC records to create proxy medical records. The method further includes creating a subset of proxy medical records that meet eligibility criteria of registration trials participants and reproducing the clinical benefits of the new drug in the subset of proxy medical records that meet the eligibility criteria of registration trials participants by adjusting one or more variables related to the clinical benefits in the analytical model and recorded in phase 3 registration trials. The method further includes applying the adjustments to the proxy medical records to generate post launch clinical benefits that persist after dilution of those adjustments by variables that were not represented in the phase 3 registration trials and accessing medical payment records for translating the post launch clinical benefits that persist into health outcome and health resource utilization.

According to another example embodiment, a system to predict reproducibility of clinical benefits of a new drug post launch of the drug is provided. The system includes non-transitory computer readable storage media configured to store one or more databases that include SOC medical records and one or more hardware processing units. The one or more hardware processing units are configured to access SOC medical records to identify a plurality of patient variables that are statistically related to one or more clinical benefits of the new drug as presented in the package insert information and to estimate strength of the identified relationships between the patient variables and the one or more clinical benefits using an analytical model. The one or more hardware processing units are configured to oversample the SOC medical records to create proxy medical records and to create a subset of proxy medical records that meet eligibility criteria of registration trials participants. The one or more hardware processing units are further configured to reproduce clinical benefits of the new drug in the subset of proxy medical records by adjusting one or more variables that are related to the clinical benefits in the analytical model and were recorded in phase 3 trials and to apply the adjustments to the SOC medical records to generate post launch clinical benefits that persist after dilution of those adjustments by the effect of variables that were not represented in the phase 3 registration trials. The one or more hardware processing units are further configured to translate the post launch clinical benefits into health outcome and health resource utilization as demonstrated in phase 3 trials of the new drug.

### BRIEF DESCRIPTION OF THE FIGURES

These and other features, aspects, and advantages of the example embodiments will become better understood when the following detailed description is read with reference to the accompanying drawings in which like characters represent like parts throughout the drawings, wherein:

FIG. 1 illustrates a system to predict reproducibility of clinical benefits of a new drug post launch in accordance with embodiments of the present technique;

FIG. 2 illustrates a method of predicting reproducibility of clinical benefits of a new drug after its launch;

FIGS. 3A-3C is an example illustration of using the system of FIG. 1 and the method of FIG. 2 to predict reproducibility of clinical benefits of a new drug;

FIG. 4 is a block diagram of an embodiment of a computing device in which the modules of the system to predict reproducibility of clinical benefits of a new drug, described herein, are implemented.

### DETAILED DESCRIPTION OF EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in many alternate forms and should not be construed as limited to only the example embodiments set forth herein.

Accordingly, while example embodiments are capable of various modifications and alternative forms, example embodiments are shown by way of example in the drawings and will herein be described in detail. It should be understood, however, that there is no intent to limit example embodiments to the particular forms disclosed. On the contrary, example embodiments are to cover all modifications, equivalents, and alternatives thereof. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently, or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. Inventive concepts may, however, be embodied in many alternate forms and should not be construed as limited to only the example embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Further, although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the scope of inventive concepts.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular example embodiments only and is not intended to be limiting. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” may encompass both an orientation of above and below. The device may be otherwise oriented (rotated 90 degrees or at other orientations) and the spatially relative descriptors used herein are interpreted accordingly.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

The device(s)/apparatus(es), described herein, may be realized by hardware elements, software elements and/or combinations thereof. For example, the devices and components illustrated in the example embodiments of inventive concepts may be implemented in one or more general-use computers or special-purpose computers, such as a processor, a controller, an arithmetic logic unit (ALU), a digital signal processor, a microcomputer, a field programmable array (FPA), a programmable logic unit (PLU), a microprocessor or any device which may execute instructions and respond. A central processing unit may implement an operating system (OS) or one or software applications running on the OS. Further, the processing unit may access, store, manipulate, process and generate data in response to execution of software. It will be understood by those skilled in the art that although a single processing unit may be illustrated for convenience of understanding, the processing unit may include a plurality of processing elements and/or a plurality of types of processing elements. For example, the central processing unit may include a plurality of processors or one processor and one controller. Also, the processing unit may have a different processing configuration, such as a parallel processor.

Software may include computer programs, codes, instructions or one or more combinations thereof and may configure a processing unit to operate in a desired manner or may independently or collectively control the processing unit. Software and/or data may be permanently or temporarily embodied in any type of machine, components, physical equipment, virtual equipment, computer storage media or units or transmitted signal waves so as to be interpreted by the processing unit or to provide instructions or data to the processing unit. Software may be dispersed throughout computer systems connected via networks and may be stored or executed in a dispersion manner. Software and data may be recorded in one or more computer-readable storage media.

The methods according to the above-described example embodiments of the inventive concept may be implemented with program instructions which may be executed by computer or processor and may be recorded in computer-readable media. The media may also include, alone or in combination with the program instructions, data files, data structures, and the like. The program instructions recorded in the media may be designed and configured especially for the example embodiments of the inventive concept or be known and available to those skilled in computer software. Computer-readable media include magnetic media such as hard disks, floppy disks, and magnetic tape; optical media such as compact disc-read only memory (CD-ROM) disks and digital versatile discs (DVDs); magneto-optical media such as floptical disks; and hardware devices that are specially configured to store and perform program instructions, such as read-only memory (ROM), random access memory (RAM), flash memory, and the like. Program instructions include both machine codes, such as produced by a compiler, and higher level codes that may be executed by the computer using an interpreter. The described hardware devices may be configured to execute one or more software modules to perform the operations of the above-described example embodiments of the inventive concept, or vice versa.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

At least one example embodiment is generally directed to techniques for proactively determining value of a drug after its launch. In particular, the embodiments are directed to techniques to leverage available clinical data to predict reproducibility of clinical benefits of a new drug after its launch. The technique further enables value-based price estimation of new drugs and biologics with transparent methodology acceptable to the payer(s), and the manufacturer of the drug.

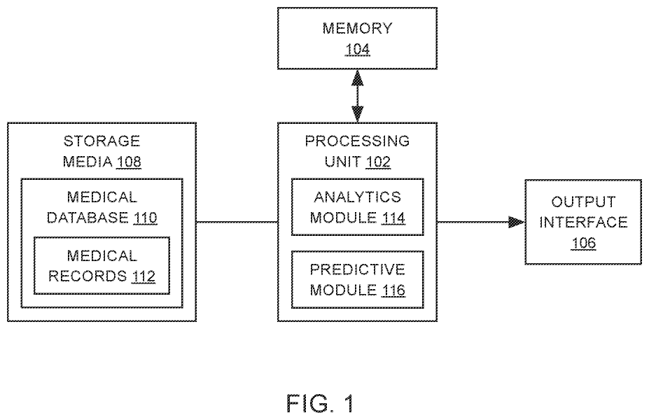

FIG. 1 illustrates a system 100 to predict reproducibility of clinical benefits of a new drug post launch of the drug in accordance with embodiments of the present technique. The system 100 includes one or more processing units such as represented by reference numeral 102, a memory 104 and an output interface 106. Each component of the system 100 is described in further detail below.

Referring again to FIG. 1, the memory 106 has computer-readable instructions stored therein, and the processing unit 102 is configured to execute the computer-readable instructions to predict reproducibility of a new drug. As used herein, the term “new drug” refers to a drug, a dietary supplement, a biologic intended to have human use and so forth. In one example, the new drug may include a drug that is recently approved by regulatory authorities. In the illustrated embodiment, the system 100 includes non-transitory computer readable storage media 108 that is configured to store one or more medical databases 110 that include standard of care (SOC) medical records 112 for a plurality of patients.

Examples of the one or more databases 110 include, but are not limited to, public and/or private healthcare organizations databases, public and/or private payers/insurers databases, public and/or private health economics and outcomes research (HEOR) databases, or combinations thereof. In some examples, the SOC medical records 112 may be stored in a plurality of databases located at multiple locations.

The processing unit 102 is communicatively coupled to the medical database 110. The processing unit 102 is configured to access the SOC medical records 112 to identify a plurality of patient variables that are statistically related to one or more clinical benefits of the new drug as presented in the package insert information of the respective drug. In one embodiment, the SOC medical records 112 correspond to medical records of treatment with a drug that can be used as a comparator, a non-pharmacological treatment that can be used as comparator, no treatment at all, or combinations thereof. The medical records 112 can include electronic medical records, laboratory results, genomic information, biospecimens, pharmacy data, social determinants of health data, or combinations there of.

In some examples, the plurality of patient variables includes risk factors of the efficacy and/or tolerability of the drug, inclusive of prescribed dosage changes and reduced exposure from non-adherence, or combinations thereof.

In this embodiment, the processing unit 102 includes an analytics module 114 configured to estimate strength of the identified relationships between the patient variables and one or more endpoints reflected in clinical benefits of the new drug using an analytical model. Moreover, the analytics module 114 is configured to oversample the SOC medical records 112 to create a first set of proxy medical records. The analytical module 114 is further configured to combine the first set of proxy medical records with the SOC records to create proxy medical records.

Moreover, the analytics module 114 is configured to create a subset of proxy medical records that meet eligibility criteria of registration trials participants. The proxy medical records and the subset of proxy medical records may be stored in the storage media 108 or in a separate storage device. The information related to the registration trials participants may be accessed from the storage media 108 or from a separate storage device.

The analytics module 114 is further configured to reproduce clinical benefits of the new drug in the subset of proxy medical records by adjusting one or more variables that are related to the clinical benefits in the analytical model and were recorded in phase 3 trials. The analytics module 114 is further configured to apply the adjustments to the SOC medical records 112 to generate post launch clinical benefits that persist after dilution of those adjustments by the effect of variables that were not represented in the phase 3 registration trials.

The processing unit 102 also includes a predictive module 116 configured to translate the post launch clinical benefits into health outcome and health resource utilization as demonstrated in phase 3 trials of the new drug. The predictive module 116 may access medical payment records for translating the post launch clinical benefits into health outcome and health resource utilization. The medical payment records may include hospital chargemaster records, medical claims, closed payer claims, or combinations thereof. Such medical payment records may be accessed from a suitable database.

In some examples, the health outcome and health resource utilization comprise increased patient survival rates, improved patient health, reduced health resource utilization, reduction or postponement of adverse experiences, or combinations thereof.

In one embodiment, the predictive module 116 is configured to analyze existing data related to the SOC drug to estimate a drug clinical effect determined to be achievable with the new drug and the health resource utilization. In another embodiment, the predictive module 116 is configured to estimate a drug price using at least one of existing data related to the new drug, predicted drug responses, estimated health care utilization of the drug, or combinations thereof.

Such health outcome and health resource utilization information may be available to a user via the output interface 106. The output interface 106 may be used to communicate estimated health outcome, health resource utilization, drug pricing to a stakeholder. The stakeholders may include a pharmaceutical organization, a pharmaceutical sponsor, a regulatory agency, an insurance company, an association of providers, or combinations thereof.

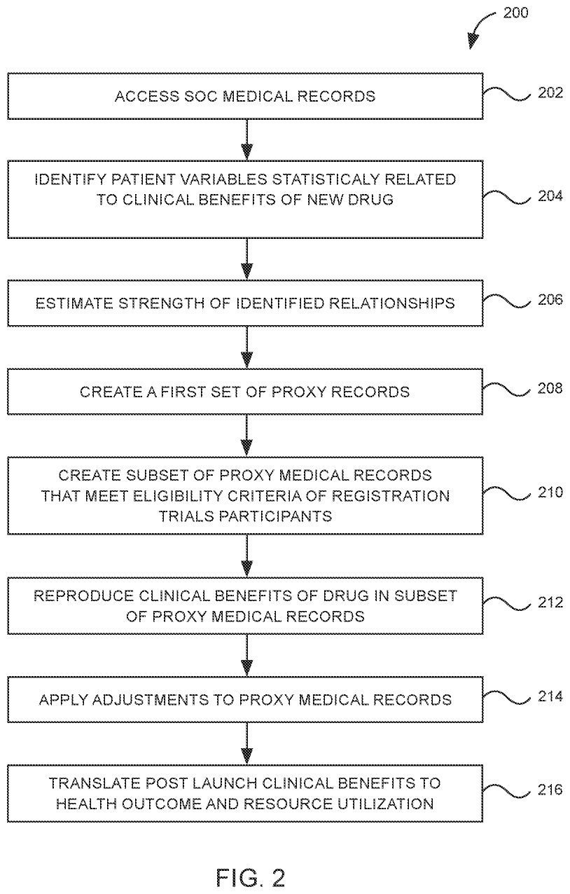

FIG. 2 illustrates a method 200 of predicting reproducibility of clinical benefits of a new drug after its launch. At block 202, standard of care (SOC) medical records is accessed. At block 204, a plurality of patient variables is identified in the SOC records that are statistically related to one or more endpoints reflected in clinical benefits of the new drug as presented in the package insert information of the drug.

Further, the strength of identified relationships between the patient variables and the one or more endpoints reflected in the clinical benefits of the new drug is estimated using an analytical model (block 206). At block 208, oversampling of the SOC medical records is performed to create a first set of proxy medical records and the first set of proxy medical records are combined with the SOC records. Further, a subset of proxy medical records that meet eligibility criteria of registration trials participants is created (block 210).

At block 212, the clinical benefits of the new drug in the subset of proxy medical records that meet the eligibility criteria of registration trials participants are reproduced by adjusting one or more variables related to the clinical benefits in the analytical model and recorded in phase 3 registration trials. Further, the adjustments are applied to the proxy medical records to generate post launch clinical benefits that persist after dilution of those adjustments by variables that were not represented in the phase 3 registration trials (block 214). At block 216, the medical payment records are accessed for translating the post launch clinical benefits that persist into health outcome and health resource utilization.

In one example, the method of predicting reproducibility of clinical benefits elicited in RCTs (before real world evidence is collected) was applied to a real situation, the recent (Aug. 7, 2020) FDA approval of a new opiate for analgesia. The drug, oliceridine (Olinvyk) has limited activity on Beta-arrestin2, a signaling pathway associated with adverse events including opiate induced respiratory distress (OIRD). In normal volunteers, morphine has about 2.5 fold greater respiratory potency compared to Olinvyk at equipotent analgesia doses. Phase 3 randomized controlled trials (RCTs) in patients treated in the ICU following surgery (bunionectomies and abdominoplasties) did not demonstrate a safety advantage (as per the statistical plan) but post hoc analyses using an OIRD definition of SpO2<90% suggested a relative risk reduction of OIRD of 23%. That is the outcome we selected for our phase 3 model.

In the illustrated example, the general method is followed to predict real world results from those observed in Phase 3 studies. An “all comers” model (big model) is fitted with all subjects eligible for the new drug (as per label) with all known covariates/response modifying factors included, and the output response taken from the RCTs (incidence of OIRD), to show the association of all known covariates with the real-world output response observed with standard of care (SOC). The “all comers” model includes all the risk factors that could have masked the advantage (safety signal) of the new drug and had been excluded in the RCTs (such as surgeries other than plastic, age over 75, any respiratory condition as per ICD-9 code, renal disease (creatinine >1.4 mg/dL, sleep apnea, and use of sedatives in the first 24 hrs of ICU admission).

Further, in the illustrated example, a small model, the Phase 3 eligible (P3E) model was fitted without the risk factors that had been excluded in the RCTs.

Further, the small model covariates were adjusted to reproduce the output response observed in RCTs. This result was achieved by reducing opiate dosage in the SOC (comparator) group to arrive at the OIRD risk reduction achieved by oliceridine as suggested by the RCT trials post-hoc analyses (−23% relative risk).

Further, the small model covariates adjustments required to reproduce the RCT trials output measures were applied to the “all comers” or big model. In the oliceridine (Olinvyk) example, this step consisted of applying the opiate dosage reduction from the third step in the small model to the big model and measure the change in output response.

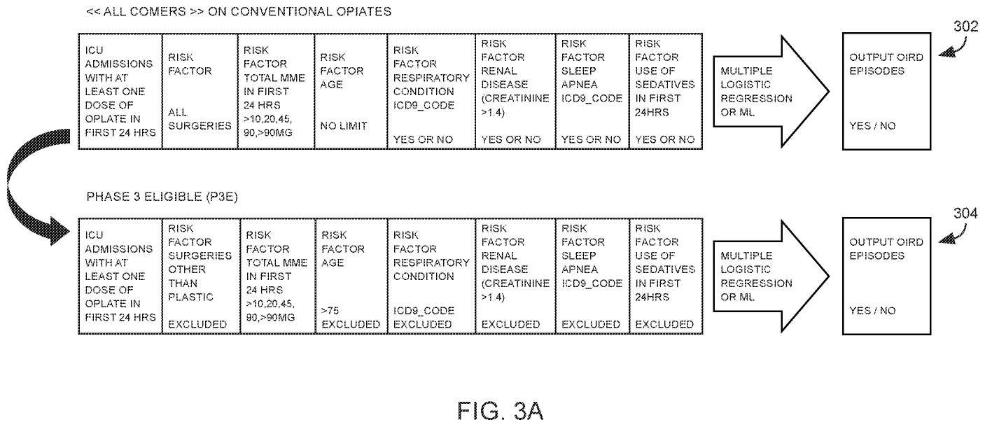

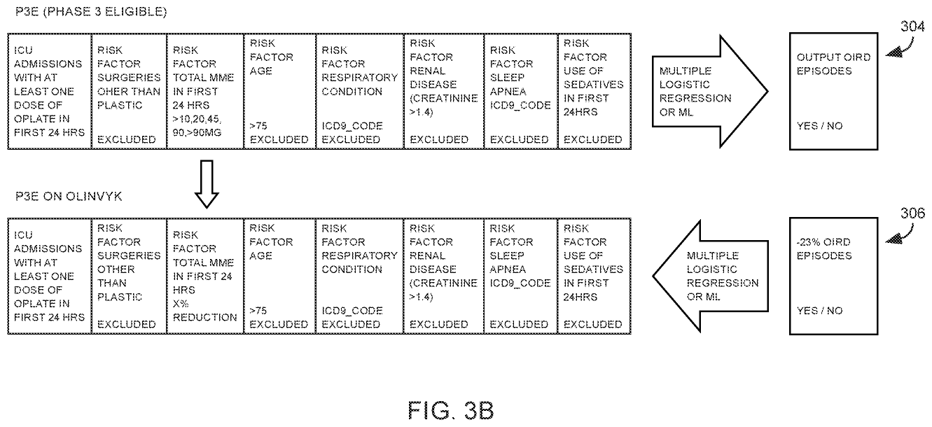

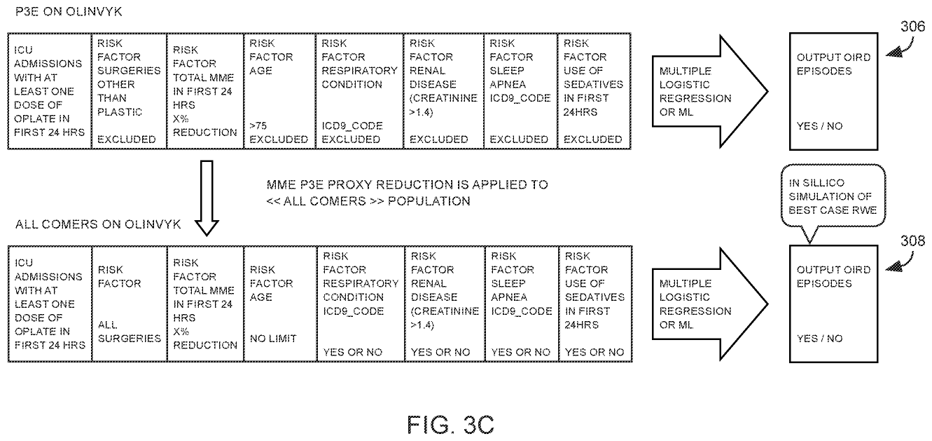

FIGS. 3A-3C is an example illustration 300 of using the system 100 of FIG. 1 and the method 200 of FIG. 2 to predict reproducibility (post-launch) of the clinical benefits of oliceridine (Olinvyk). As can be seen in blocks 302 and 304, phase 3 eligible (P3E) subjects were selected out of a group of “all comers”. “All comers” are patients with moderate-to-severe pain following any surgical intervention. Phase 3 eligible (P3E) subjects had been defined (by the sponsor) as a sub-group of “all comers” for whom no other surgery had been allowed and several risk factors (such as old age, respiratory conditions, renal disease, sleep apnea and use of sedatives in the first 24 hours) had been excluded to eliminate possible confounders in the interpretation of clinical results.

As illustrated in blocks 304 to 306, the benefits of the experimental drug (Olinvyk) over standard of care (morphine and other traditional opiates) were reproduced (mathematically) by reducing the dose of opiates (calculated in morphine-milligram-equivalent, or MME), which was associated with respiratory complications and a modifiable covariate in the logistic regression model to achieve the OIRD rate reduction (−23%) achieved with Olinvyk. In other words, in this example, the reduced dose of morphine was used as a proxy for substituting “standard of care” (SOC) drug(s) (morphine and traditional opiates) by the new drug (Olinvyk) in a simulated Phase 3 trial. Further, as illustrated in blocks 306 to 308, the P3E proxy dose reduction in morphine was applied to the entire group of “all comers”.

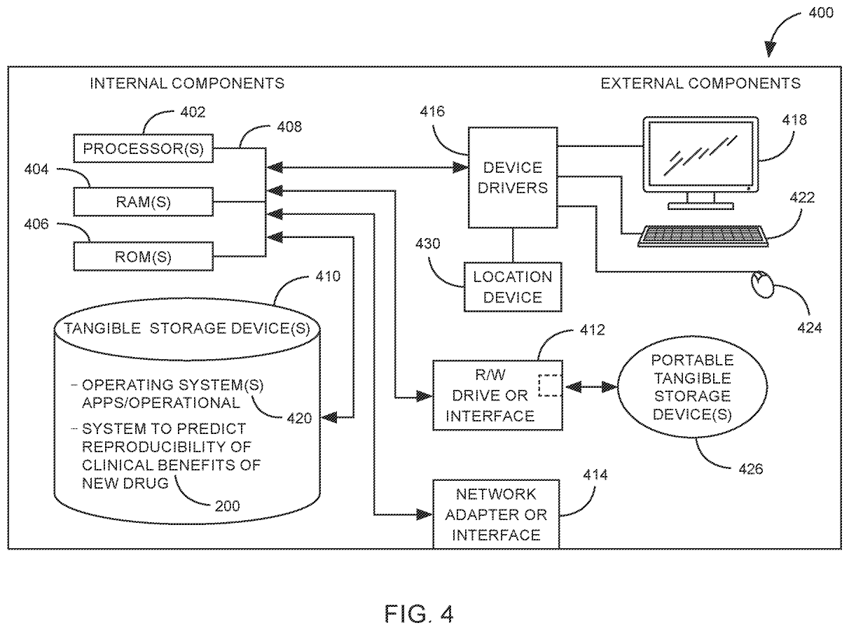

The modules of the system 100 described herein are implemented in computing devices. One example of a computing device 400 is described below in FIG. 4. The computing device includes one or more processor 402, one or more computer-readable RAMs 404 and one or more computer-readable ROMs 406 on one or more buses 408. Further, computing device 400 includes a tangible storage device 410 that may be used to execute operating systems 420 and the system 100 to predict reproducibility of clinical benefits of a new drug. The various modules of the system 100 may be stored in tangible storage device 410. Both, the operating system 420 and the system 100 are executed by processor 402 via one or more respective RAMs 404 (which typically include cache memory). The execution of the operating system 420 and/or the system 100 by the processor 402, configures the processor 402 as a special purpose processor configured to carry out the functionalities of the operation system 420 and/or the system 100, as described above.

Examples of storage devices 410 include semiconductor storage devices such as ROM 406, EPROM, flash memory or any other computer-readable tangible storage device that may store a computer program and digital information.

Computing device also includes a R/W drive or interface 414 to read from and write to one or more portable computer-readable tangible storage devices 428 such as a CD-ROM, DVD, memory stick or semiconductor storage device. Further, network adapters or interfaces 412 such as a TCP/IP adapter cards, wireless Wi-Fi interface cards, or 3G or 4G wireless interface cards or other wired or wireless communication links are also included in computing device.

In one example embodiment, the system 100 which includes a processing unit 102 and a memory 106, may be stored in tangible storage device 410 and may be downloaded from an external computer via a network (for example, the Internet, a local area network or other, wide area network) and network adapter or interface 412.

Computing device further includes device drivers 416 to interface with input and output devices. The input and output devices may include a computer display monitor 418, a keyboard 424, a keypad, a touch screen, a computer mouse 426, and/or some other suitable input device.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

While only certain features of several embodiments have been illustrated, and described herein, many modifications and changes will occur to those skilled in the art. It is, therefore, to be understood that the appended claims are intended to cover all such modifications and changes as fall within the true spirit of inventive concepts.

The aforementioned description is merely illustrative in nature and is in no way intended to limit the disclosure, its application, or uses. The broad teachings of the disclosure may be implemented in a variety of forms. Therefore, while this disclosure includes particular examples, the true scope of the disclosure should not be so limited since other modifications will become apparent upon a study of the drawings, the specification. It should be understood that one or more steps within a method may be executed in different order (or concurrently) without altering the principles of the present disclosure. Further, although each of the example embodiments is described above as having certain features, any one or more of those features described with respect to any example embodiment of the disclosure may be implemented in and/or combined with features of any of the other embodiments, even if that combination is not explicitly described. In other words, the described example embodiments are not mutually exclusive, and permutations of one or more example embodiments with one another remain within the scope of this disclosure.

The example embodiment or each example embodiment should not be understood as a limiting/restrictive of inventive concepts. Rather, numerous variations and modifications are possible in the context of the present disclosure, in particular those variants and combinations which may be inferred by the person skilled in the art with regard to achieving the object for example by combination or modification of individual features or elements or method steps that are described in connection with the general or specific part of the description and/or the drawings, and, by way of combinable features, lead to a new subject matter or to new method steps or sequences of method steps, including insofar as they concern production, testing and operating methods. Further, elements and/or features of different example embodiments may be combined with each other and/or substituted for each other within the scope of this disclosure.

Still further, any one of the above-described and other example features of example embodiments may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Further, at least one example embodiment relates to a non-transitory computer-readable storage medium comprising electronically readable control information (e.g., computer-readable instructions) stored thereon, configured such that when the storage medium is used in a controller of a magnetic resonance device, at least one example embodiment of the method is carried out.

Even further, any of the aforementioned methods may be embodied in the form of a program. The program may be stored on a non-transitory computer readable medium, such that when run on a computer device (e.g., a processor), cause the computer-device to perform any one of the aforementioned methods. Thus, the non-transitory, tangible computer readable medium, is adapted to store information and is adapted to interact with a data processing facility or computer device to execute the program of any of the above mentioned embodiments and/or to perform the method of any of the above mentioned embodiments.

The computer readable medium or storage medium may be a built-in medium installed inside a computer device main body or a removable medium arranged so that it may be separated from the computer device main body. The term computer-readable medium, as used herein, does not encompass transitory electrical or electromagnetic signals propagating through a medium (such as on a carrier wave), the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices), volatile memory devices (including, for example static random access memory devices or a dynamic random access memory devices), magnetic storage media (including, for example an analog or digital magnetic tape or a hard disk drive), and optical storage media (including, for example a CD, a DVD, or a Blu-ray Disc). Examples of the media with a built-in rewriteable non-volatile memory, include but are not limited to memory cards, and media with a built-in ROM, including but not limited to ROM cassettes, etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave), the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices), volatile memory devices (including, for example static random access memory devices or a dynamic random access memory devices), magnetic storage media (including, for example an analog or digital magnetic tape or a hard disk drive), and optical storage media (including, for example a CD, a DVD, or a Blu-ray Disc). Examples of the media with a built-in rewriteable non-volatile memory, include but are not limited to memory cards, and media with a built-in ROM, including but not limited to ROM cassettes, etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which may be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

1. A computer-implemented method of predicting reproducibility of clinical benefits of a new drug after its launch, the method comprising:
accessing standard of care (SOC) medical records;
identifying in SOC records a plurality of patient variables that are statistically related to one or more endpoints reflected in clinical benefits of the new drug as presented in the package insert information of the drug;
estimating the strength of identified relationships between the patient variables and the one or more endpoints reflected in the clinical benefits of the new drug using an analytical model;
oversampling the SOC medical records to create a first set of proxy medical records;
combining the first set of proxy medical records with the SOC records to create proxy medical records;
creating a subset of proxy medical records that meet eligibility criteria of registration trials participants;
reproducing the clinical benefits of the new drug in the subset of proxy medical records that meet the eligibility criteria of registration trials participants by adjusting one or more variables related to the clinical benefits in the analytical model and recorded in phase 3 registration trials;
applying the adjustments to the proxy medical records to generate post launch clinical benefits that persist after dilution of those adjustments by variables that were not represented in the phase 3 registration trials; and
accessing medical payment records for translating the post launch clinical benefits that persist into health outcome and health resource utilization.

2. The computer implemented method of claim 1, wherein the medical records comprise electronic medical records, laboratory results, genomic information, biospecimens, pharmacy data, social determinants of health data, or combinations there of.

3. The computer implemented method of claim 1, wherein the new drug comprises a drug that is recently approved by regulatory authorities.

4. The computer-implemented method of claim 1, wherein the SOC medical records correspond to medical records of treatment with a drug that can be used as a comparator, a non-pharmacological treatment that can be used as comparator, no treatment at all, or combinations thereof.

5. The computer-implemented method of claim 1, comprising accessing medical payment records from public and/or private healthcare databases, or combinations thereof.

6. The computer-implemented method of claim 1, wherein the patient variables comprise risk factors of the efficacy and tolerability of the SOC drug, wherein the factors comprise medically prescribed dosage adjustments of the SOC drug, changes in exposure from non-adherence, or combinations thereof.

7. The computer-implemented method of claim 1, wherein translating the post launch clinical benefits further comprises analyzing existing data related to the SOC drug to estimate a drug clinical effect determined to be achievable with the new drug and the health resource utilization.

8. The computer-implemented system of claim 7, further comprising estimating a drug price using at least one of existing data related to the new drug, predicted drug responses, estimated health care utilization of the drug, or combinations thereof.

9. The computer-implemented system of claim 7, wherein the one or more clinical benefits of the new drug comprises one or more benefits listed on a label of the drug.

10. The computer-implemented method of claim 1, wherein the medical payment records comprise hospital chargemaster records, medical claims, closed payer claims, or combinations thereof.

11. A system to predict reproducibility of clinical benefits of a new drug post launch of the drug, wherein the system comprises:
non-transitory computer readable storage media configured to store one or more databases that include SOC medical records; and
one or more hardware processing units configured to:
access SOC medical records to identify a plurality of patient variables that are statistically related to one or more clinical benefits of the new drug as presented in the package insert information;
estimate strength of the identified relationships between the patient variables and the one or more clinical benefits using an analytical model;
oversample the SOC medical records to create proxy medical records;
create a subset of proxy medical records that meet eligibility criteria of registration trials participants;
reproduce clinical benefits of the new drug in the subset of proxy medical records by adjusting one or more variables that are related to the clinical benefits in the analytical model and were recorded in phase 3 trials;
apply the adjustments to the SOC medical records to generate post launch clinical benefits that persist after dilution of those adjustments by the effect of variables that were not represented in the phase 3 registration trials; and
translate the post launch clinical benefits into health outcome and health resource utilization as demonstrated in phase 3 trials of the new drug.

12. The system of claim 11, wherein the one or more databases comprise health economics and outcomes research (HEOR), a public and/or private healthcare database, or combinations thereof.

13. The system of claim 11, wherein the patient variables comprise risk factors of the efficacy and/or tolerability of the drug, inclusive of prescribed dosage changes and reduced exposure from non-adherence, or combinations thereof.

14. The system of claim 11, wherein the one or more databases is configured to store the SOC medical records and the proxy medical records.

15. The system of claim 14, wherein the SOC medical records correspond to medical records of treatment with a drug that can be used as a comparator, a non-pharmacological treatment that can be used as comparator, no treatment at all, or combinations thereof.

16. The system of claim 11, wherein the new drug comprises a drug, a dietary supplement, a biologic intended to have a human use.

17. The system of claim 11, wherein the system comprises an output interface to communicate estimated health outcome, health resource utilization, drug pricing to a stakeholder.

18. The system of claim 17, wherein the stakeholder comprises a pharmaceutical organization, a pharmaceutical sponsor, a regulatory agency, an insurance company, an association of providers, or combinations thereof.

19. The system of claim 11, wherein the health outcome and health resource utilization comprise increased patient survival rates, improved patient health, reduced health resource utilization, reduction or postponement of adverse experiences, or combinations thereof.

20. The system of claim 11, wherein the SOC medical records are stored in a plurality of databases located at multiple locations.
